(19) United States
(12) Patent Application Publication
(10) Pub. No.: US 2005/0119179 A1
(43) Pub. Date: Jun. 2, 2005

(54) Inositol 1, 4, 5-triphosphate receptor (type 1), phosphorylation and modulation by CDC2

(76) Inventor: Thottala Jayaraman, Ardsley, NY (US)

Correspondence Address: LESLIE GLADSTONE RESTAINO;BROWN RAYSMAN MILLSTEIN & STEINER LLP, 163 MADISON AVENUE, PO BOX 1989, MORRISTOWN, NJ, 07962-1989, US

(21) Appl. No.: 10/899,639

(22) Filed: Jul. 26, 2004

Related U.S. Application Data
(60) Provisional application No. 60/490,380, filed on Jul. 25, 2003.

(51) Int. Cl. A61K 38/17

(52) U.S. Cl. 514/12

(57) ABSTRACT

The present invention relates generally to identification of inositol 1,4,5-triphosphate receptor 1 as a target for cdc2/CyB during cell cycle progression. The invention provides molecules and compositions that can be used to regulate intracellular calcium, cell cycle progression, mitotic catastrophe, apoptosis and cellular death processes. The invention also provides methods and kits for regulating cellular death in individual disease processes.

### RELATED APPLICATION

This application claims the benefit of U.S. Provisional Application No. 60/490,380, filed Jul. 25, 2003.

### FIELD OF THE INVENTION

The present invention relates generally to identification of inositol 1,4,5-triphosphate receptor 1 as a target for cdc2/CyB during cell cycle progression. The invention provides molecules and compositions that can be used to regulate intracellular calcium, cell cycle progression, mitotic catastrophe, apoptosis and cellular death processes. The invention also provides methods and kits for regulating cellular death in individual disease processes.

### BACKGROUND OF THE INVENTION

IP3R-mediated Ca²⁺ signaling is involved in modulating cell growth and death pathways (Jayaraman and Marks 1997; Marks 1997), and IP3Rs are ubiquitously expressed intracellular Ca²⁺-release channels in many cell types (Ehrlich 1994; Marks 1997). In mammalian tissues, at least three forms of IP3R have been identified. The channel exists as homotetrameric and heterotetrameric structures (Joseph, et al., 1995; Nucifora, et al., 1996) with three functional domains: a transmembrane domain containing the Ca²⁺-channel pore close to the carboxy-terminus, the amino-terminal IP3-binding domain, and a large cytosolic domain that connects the Ca²⁺ channel with the IP3-binding region (Mignery 1990; Joseph 1996). Most of the IP3Rs, excluding a short transmembrane Ca²⁺-channel region, are exposed to the cytoplasm and are targets for several accessory proteins as well as kinases. For instance, IP3R1 functions are modulated by several accessory proteins including the FK-506 binding protein, FKBP12, a member of the immunophilin family of cis-trans peptidylprolyl isomerases (Cameron, et al., 1995b; Poirier, et al., 2001), calcineurin (Cameron, et al., 1995a), homer protein that binds to a proline-rich motif (Tu, et al., 1998), the non-receptor protein tyrosine kinase Fyn (Jayaraman, et al., 1996), and inositol 1,4,5-trisphosphate receptor-associated cGMP substrate (IRAG) (Schlossmann, et al., 2000). A homer ligand-like motif is conserved in IP3R1 at amino acids 48-55; the binding regions for Fyn and PKG have not yet been determined.

In addition to accessory proteins, five protein kinases, Fyn (Jayaraman, et al., 1996), calmodulin-dependent kinase II (CaMKII) (Zhu, et al., 1996), protein kinase G (PKG) (Schlossmann, et al., 2000), protein kinase A (PKA) (Nakade, et al., 1994), and protein kinase C (PKC) (Matter, et al., 1993) modulate IP3R1 via phosphorylation. Although the precise details of how phosphorylation by specific kinases modulates IP3R1 function, and the significance of these signaling events in the context of cellular function, remain to be elucidated, there are well-documented functional effects that are observed upon phosphorylation of IP3R1 (Matter, et al., 1993; Komalavilas 1994; Nakade, et al., 1994).

Ca²⁺ transients occur when cells progress from quiescence, at the G1/S transition, during S-phase, and at the exit from mitosis (Berridge 1995), as the resulting Ca²⁺ signals are required to initiate many types of transcriptional events during cellular proliferation. Ca²⁺ transients are elicited in the form of oscillations during the cell cycle in dividing Xenopus embryos and in cycling egg extracts (Poenie, et al., 1985; Grandin 1991; Kubota, et al., 1993; Swanson, et al., 1997; Tokmakov, et al., 2001). The finding that injection of an antibody specific to IP3R1 blocks Ca²⁺ oscillations in fertilized hamster eggs suggests the involvement of IP3R1 (Miyazaki, et al., 1992). Injection of Ca²⁺ chelators into fertilized eggs blocks cell cycle progression, whereas Ca²⁺ ionophores induce its resumption. Microinjection of BAPTA, an intracellular Ca²⁺ chelator, prevents mitosis in Xenopus embryos (which express only IP3R1) and inhibits proliferation of mesangial cells in response to PDGF, endothelin-1, and FBS (Whiteside, et al., 1998). Moreover, an IP3R1 deletion mutant lacking the IP3-binding region and IP3R1-deficient T cells (generated using 2.9 kb of 5′ antisense DNA) display reduced cell growth in response to serum (Fischer, et al., 1994; Jayaraman and Marks 1997). In addition, a recent report demonstrates a crucial role of IP3R1, but not IP3R3, in IP3-induced Ca²⁺ release and proliferation of vascular smooth muscle cells (Wang, et al., 2001). These studies suggest that IP3R1-mediated Ca²⁺ release is important for cellular proliferation; however, the molecular mechanism by which IP3R1 functions are modulated during cell cycle progression is not known.

Orderly progression through the cell cycle depends on the activation and inactivation of cdks. While cdc2/CyB (also known as the cdk1/CyB complex) is necessary for G2/M transition, cdk4/CyD, cdk2/CyE and cdk2/CyA play major roles in the G1, G1/S and S-phase transitions of the cell cycle, respectively (Morgan 1997). Each of these cdks is active for only a short period of the cell cycle, during which time it phosphorylates a number of substrates required for entry into the next phase. The inventor has examined IP3R1 phosphorylation by the cdc2/CyB complex in vitro and determined the effect of phosphorylation on IP3 binding.

The present invention demonstrates that cdc2/CyB-mediated phosphorylation of IP3Rs positively regulate Ca²⁺ with increased sensitivity to activation-induced apoptosis. Accordingly, persistent or untimely activation of cdks, as in HIV and neurodegenerative diseases such as Alzheimer's disease, may be lethal to the cell because it modulates IP3R sensitivity to IP3, resulting in increased Ca²⁺ release, which is perceived by the cell as an apoptotic signal. The inventor's identification of a CyB docking site on IP3R1 demonstrates, for the first time, direct interaction between a cell cycle component and an intracellular calcium release channel. Cdc2-mediated phosphorylation of IP3Rs, resulting in intracellular calcium release, is likely a specific shared step critical to cell cycle progression, mitotic catastrophe and apoptosis.

In view of the prior art, a need remains to identify compounds and methods that can be used to selectively regulate cellular death processes. In particular, there is an important need to be able to selectively target and to induce cellular death in certain cells, such as tumor cells. Similarly, there is a critical need to be able to selectively block or prevent cellular death or apoptosis involved in certain diseases. For example, selectively blocking phosphorylation of IP3Rs in specific neurons in a subject suffering from a neurodegenerative disease such as Alzheimer's disease or Parkinson's disease, could ameliorate the disease by selectively blocking apoptosis in these cells. Cellular death in CD4+ helper T cells affected by HIV or AIDS could similarly be blocked by selectively blocking phosphorylation of IP3Rs in these cells.

### SUMMARY OF THE INVENTION

Calcium (Ca²⁺) release from the endoplasmic reticulum (ER) controls numerous cellular functions including proliferation and is regulated in part by Inositol 1,4,5-trisphosphate receptors (IP3Rs). IP3Rs are ubiquitously expressed intracellular Ca²⁺-release channels found in many cell types. Although IP3R-mediated Ca²⁺ release has been implicated in cellular proliferation, the biochemical pathways that modulate intracellular Ca²⁺ release during cell cycle progression are not known. Sequence analysis of IP3R1 reveals the presence of two putative phosphorylation sites for cyclin-dependent kinases (cdks). The inventor has shown for the first time that cdc2/CyB, a critical regulator of eukaryotic cell cycle progression, phosphorylates IP3R1 in vitro and in vivo at both Ser⁴²¹ and Thr⁷⁹⁹ and that this phosphorylation increases IP3 binding. This phosphorylation is mediated by direct binding of CyB to IP3R1 through Arg³⁹¹, Arg⁴⁴¹ and Arg⁸⁷¹ and is increased during activation-induced apoptosis. The inventor has also shown that cdc2-mediated phosphorylation greatly increases the binding of IP3 to IP3R1. Taken together, these results indicate that IP3R1 is a specific target for cdc2/CyB during cell cycle progression.

The inventor has further shown that cdc2/CyB complex phosphorylates IP3R3 in vitro and in vivo. These results indicate that cdc2/CyB-mediated phosphorylation of IP3Rs may positively regulate IP3-gated Ca2+ with increased sensitivity to activation-induced apoptosis.

The present invention provides compositions and methods for regulating intracellular calcium, cell cycle progression, mitotic catastrophe, apoptosis and cellular death processes. In one aspect, the invention provides a composition for use in increasing intracellular calcium comprising an IP3R1 agonist. In one embodiment of the present invention, the agonist enhances phosphorylation of IP3R1 at Ser⁴²¹ and Thr⁷⁹⁹. In another embodiment, the agonist enhances CyB binding to IP3R1 at Arg³⁹¹, Arg⁴⁴¹ and Arg⁸⁷¹.

The invention also provides compositions for use in decreasing intracellular calcium comprising an IP3R1 antagonist. In an embodiment of the present invention, the antagonist prevents the phosphorylation of IP3R1 at Ser⁴²¹ and Thr⁷⁹⁹. In another embodiment of the invention, the antagonist prevents or inhibits CyB binding to IP3R1 at Arg³⁹¹, Arg⁴⁴¹ and Arg⁸⁷¹.

The present invention further provides methods for preventing cell death comprising administering to cells an effective amount of an IP3R1 antagonist, wherein the antagonist prevents or inhibits phosphorylation of IP3R1. A method for treating or preventing a disease involving cell death in a subject is also provided which comprises administering to a subject a therapeutically effective amount of an IP3R1 antagonist, wherein the antagonist prevents or inhibits phosphorylation of IP3R1. In one embodiment of the invention, the disease treated or prevented is a neurodegenerative disease. In a further embodiment, the neurodegenerative disease is selected from the group consisting of: Alzheimer's disease, Parkinson's disease, Huntington's disease, amyotrophic lateral sclerosis, Pick's disease, progressive supranuclear palsy and corticobasal degeneration. In yet a further embodiment of the invention, the disease treated or prevented is HIV or AIDS.

The present invention additionally provides methods for inducing cell death. In an embodiment, a method for inducing cell death is provided that comprises administering to the cell an effective amount of an IP3R1 agonist, wherein the agonist enhances phosphorylation of IP3R1. In another embodiment, the cells are tumor cells.

The present invention also provides a method for treating or preventing a proliferative disease comprising administering to a subject in need thereof a therapeutically effective amount of a composition comprising an IP3R1 agonist, wherein the agonist enhances phosphorylation of IP3R1. In one embodiment of the present invention, the proliferative disease is cancer.

The invention further provides kits for use in regulating intracellular calcium and treating or preventing various disorders modulated by IP3R activity. In one embodiment, a kit for treating and preventing neurodegenerative disease is provided, comprising an effective amount of an IP3R1 antagonist. In another embodiment, a kit for use in treating and preventing HIV infection comprising an effective amount of an IP3R1 antagonist is provided. In a further embodiment, the invention provides a kit for use in treating and preventing cancer comprising an effective amount of an IP3R1 agonist.

### DESCRIPTION OF THE FIGURES

FIG. 1 Cdks phosphorylate IP3R1 in vitro. A. Sequence alignment of cdk phosphorylation motifs in IP3Rs and species. The letters denote: m (mouse), r (rat), h (human) and x (Xenopus). GenBank accession numbers are: mIP3R1 (X15373); rIP3R1 (J005510); hIP3R1 (L38019); and xIP3R1 (D14400). B. In vitro kinase reactions with ³²P γ[ATP] were performed in the presence of cdc2/CyB kinase with αIP3R1 immunoprecipitates from brain microsomes made using either normal rabbit serum (NRS) or αIP3R1 antibody. C. The cdk inhibitor roscovitine (100 nM) inhibited phosphorylation of IP3R1. The negative control was immunoprecipitation without αIP3R1 antibody.

FIG. 2 Cdc2/CyB phosphorylates IP3R1 at Ser⁴²¹ and Thr⁷⁹⁹ in vitro. WT and mutant (S421A and T799A) GST-IP3R1 fragments were expressed. A. Schematic representation of WT and mutant constructs used for the experiments. B. Immunoblot showing the input GST-fusion proteins for IP3R1/375473 and the mutant IP3R1/375-473/S421A. C. Immunoblot showing the input GST-fusion proteins for IP3R1/753-886, and the mutant IP3R1/753-886/T799A. D. In vitro kinase reactions performed with [γ³²P] ATP and cdc2/CyB.

FIG. 3 αS421 and αT799 specifically recognize phosphorylated IP3R1 peptides and native protein. Non-phosphorylated and phosphorylated IP3R1 peptides were blotted at the indicated concentration and immunoblotted with 1:1,000 dilution of αSer⁴²¹ (A) and αThr⁷⁹⁹ antibody (B). The in vitro kinase reactions were performed with 200 ng of fusion proteins with 2 units of cdc2/CyB complex with and without cdc2/CyB in kinase buffer at 30° C. for 10 min. The reaction was terminated by the addition of 3× sample buffer and heating to 95° C. for 5 min. The proteins were transferred to nitrocellulose membrane and probed with 1:1,000 dilution of αSer⁴²¹ antibody (C) and developed with ECL reagents. The blot was then stripped and probed with 1:1,000 dilution of αThr⁷⁹⁹ antibody (D). In vitro kinase reactions were performed from IP3R1 immunoprecipitates with and without cdc2/CyB and immunoblotted with αSer⁴²¹ antibody (E), αThr⁷⁹⁹ antibody (F) and αIP3R1 (G).

FIG. 4 IP3R1 phosphorylation at Ser421 and Thr799 occurs in vivo. DT40 cells were serum-starved for 24 h and cultured with and without 1 μM nocodazole (control) for 16 h in RPMI-1640 medium containing 10% FBS. After 16 h in culture, cells were washed two times with PBS, and cell lysates were resolved on 6% SDS-PAGE gels and immunoblotted with αSer⁴²¹ (A), αThr⁷⁹⁹ (B) or αIP3R1 (C).

FIG. 5 Cdc2/CyB phosphorylation of IP3R1 increases IP3 binding. A. The soluble proteins (30 μg) from E. coli expressing IP3R1(1-900) were either left alone or phosphorylated with cdc2/CyB complex. Controls included E. coli proteins from vector alone and the inclusion of roscovitine, a cdc2 inhibitor. The inclusion of roscovitine completely blocked the phosphorylation by cdc2/CyB complex. B. [³H]IP3 binding to soluble proteins (30 μg) from E. coli transformed with pGEX-IP3R1(1-900), phosphorylated with cdc2/CyB, and phosphorylated with cdc2 in the presence of roscovitine (cdc2 inhibitor) and pGEX-2T (vector). Nonspecific binding was measured in the presence of 2 μM IP3. Values are the means ±S.D. of three separate experiments. Identical parallel phosphorylation reactions were set up for detecting phosphorylation (A) and IP3 binding (B).

FIG. 6 2-APB and Xestospongin affect IP3 receptor function of phosporylation deficient mutant before and after activation. The compounds were tested on wild type DT40 cells. The phosphorylation deficient cells were generated by transfecting phosphorylation deficient IP3R mutant cDNAs into triple IP3R deficient DT40 cells. These cells express mutant IP3R with Ser421 and Thr799 replaced by alanine residues. The remaining portion of the receptor is the same as native IP3R. The lack of apoptosis after stimulation suggests that phosphorylation is necessary for apoptosis induction.

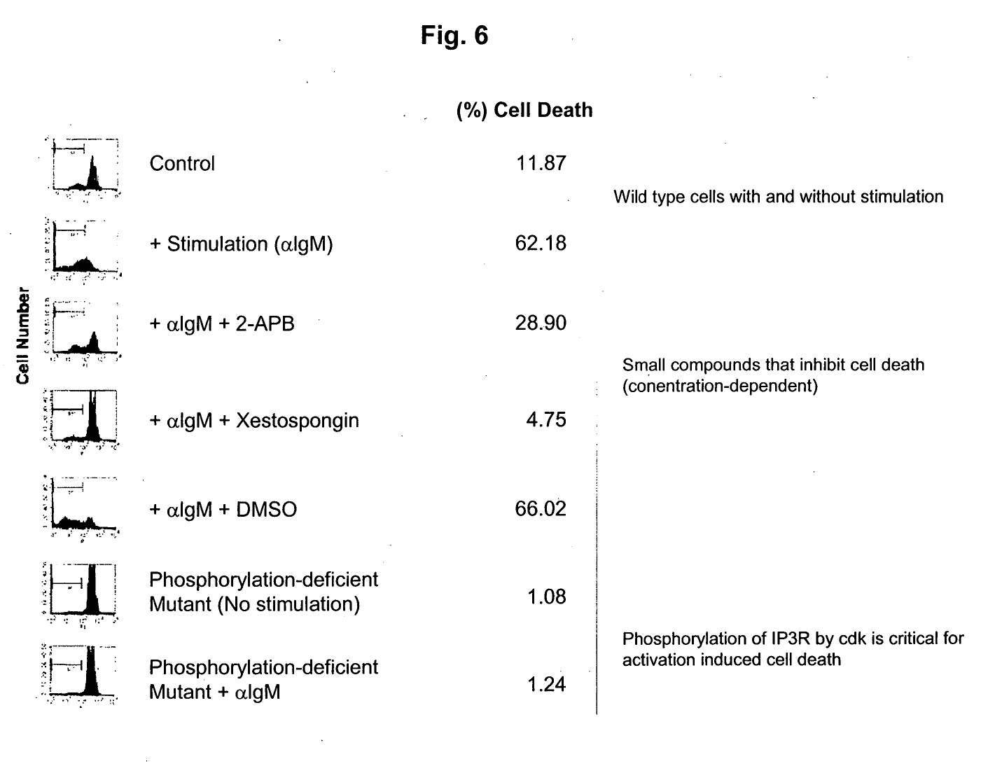

### DETAILED DESCRIPTION OF THE INVENTION

The present invention relates generally to identification of inositol 1,4,5-triphosphate receptor 1 as a target for cdc2/CyB during cell cycle progression. The invention provides molecules and compositions that can be used to regulate intracellular calcium, cell cycle progression, mitotic catastrophe, apoptosis and cellular death processes. The invention additionally provides methods and kits for regulating cellular death in individual disease processes.

Specifically, the present invention encompasses compositions, including pharmaceutical compositions, for use in increasing intracellular calcium. In one embodiment of the invention, the composition comprises an IP3R agonist, such as, for example, an IP3R1 agonist. In another embodiment of the invention, the agonist enhances phosphorylation of IP3R1 at Ser⁴²¹ and Thr⁷⁹⁹. In still another embodiment of the present invention, the agonist enhances CyB binding to IP3R1.

As used in accordance with the present invention, the term “IP3R agonist” refers to IP3R agonists and analogues and derivatives thereof, including, for example, natural or synthetic functional variants which have IP3R biological activity, as well as fragments of an IP3R agonist having IP3R biological activity. As further used herein, the term “IP3R biological activity” refers to activity that mimics the effects of cdc2/CyB in a subject and which enhances or improves phosphorylation of IP3R1 at Ser⁴²¹ and/or Thr⁷⁹⁹. IP3R biological activity also refers to activity which enhances or improves the binding of CyB to IP3R1 at Arg³⁹¹, Arg⁴⁴¹ and Arg⁸⁷¹. Commonly known IP3R agonists include, but are not limited to, the cdc2/CyB (cdk1/CyB) complex as well as other similar chemical substances, peptides or small molecules capable of combining with a the IP3R1 receptor and initiating IP3R biological activity.

The present invention further encompasses compositions, including pharmaceutical compositions, for use in decreasing intracellular calcium. In one embodiment of the invention, the composition comprises an IP3R antagonist, such as, for example, an IP3R1 antagonist. In another embodiment of the invention, the agonist prevents, blocks or inhibits phosphorylation of IP3R1 at Ser⁴²¹ and/or Thr⁷⁹⁹. In still another embodiment of the present invention, the antagonist prevents or inhibits CyB binding to IP3R1.

As used in accordance with the present invention, “IP3R antagonist” refers to IP3R antagonists and analogues and derivatives thereof including, for example, natural or synthetic functional variants which have IP3R antagonist biological activity, as well as fragments of an IP3R antagonist having IP3R antagonist biological activity. As further used herein, the term “IP3R antagonist biological activity” refers to activity that prevents, blocks or inhibits phosphorylation of IP3R. IP3R antagonist biological activity also refers to activity which inhibits or prevents the binding of CyB to IP3R1 at Arg³⁹¹, Arg⁴⁴¹ and Arg⁸⁷¹. IP3R antagonists include, but are not limited to, chemical substances, peptides or other small molecules capable of combining with a the IP3R1 receptor and initiating IP3R antagonist biological activity.

IP3R agonists and IP3R antagonists may be synthesized or otherwise prepared in accordance with known procedures that are readily understood by those of skill in the art.

The therapeutic agents of the present invention (i.e., the IP3R agonist and the IP3R antagonist, either in separate, individual formulations or in a single, combined formulation) may be administered to a human or animal subject by known procedures including, but not limited to, oral administration, parenteral administration (e.g., intramuscular, intraperitoneal, intravascular, intravenous or subcutaneous administration) and transdermal administration. Preferably, the therapeutic agents of the present invention are administered orally or intravenously.

For oral administration, the formulations of the IP3R agonist, as well as the IP3R antagonist may be presented as capsules, tablets, powders, granules, or as a suspension. The formulations may have conventional additives, such as lactose, mannitol, corn starch or potato starch. The formulations also may be presented with binders, such as crystalline cellulose, cellulose derivatives, acacia, corn starch or gelatins. Additionally, the formulations may be presented with disintegrators, such as corn starch, potato starch or sodium carboxymethyl cellulose. The formulations also may be presented with dibasic calcium phosphate anhydrous or sodium starch glycolate. Finally, the formulations may be presented with lubricants, such as talc or magnesium stearate.

For parenteral administration, the formulations of the IP3R agonist as well as the IP3R antagonist may be combined with a sterile aqueous solution which is preferably isotonic with the blood of the subject. Such formulations may be prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulations may be presented in unit or multi-dose containers, such as sealed ampules or vials. Moreover, the formulations may be delivered by any mode of injection including, without limitation, epifascial, intracapsular, intracutaneous, intramuscular, intraorbital, intraperitoneal (particularly in the case of localized regional therapies), intraspinal, intrasternal, intravascular, intravenous, parenchymatous or subcutaneous.

For transdermal administration, the formulations of the IP3R agonist and the IP3R antagonist may be combined with skin penetration enhancers, such as propylene glycol, polyethylene glycol, isopropanol, ethanol, oleic acid, N-methylpyrrolidone and the like, which increase the permeability of the skin to the therapeutic agent and permit the therapeutic agent to penetrate through the skin and into the bloodstream. The therapeutic agent/enhancer compositions also may be further combined with a polymeric substance, such as ethylcellulose, hydroxypropyl cellulose, ethylene/vinylacetate, polyvinyl pyrrolidone and the like, to provide the composition in gel form, which may be dissolved in a solvent such as methylene chloride, evaporated to the desired viscosity and then applied to backing material to provide a patch.

The dose of the IP3R agonists and the IP3R antagonists of the present invention may also be released or delivered from an osmotic mini-pump. The release rate from an elementary osmotic mini-pump may be modulated with a microporous, fast-response gel disposed in the release orifice. An osmotic mini-pump would be useful for controlling release, or targeting delivery, of the therapeutic agents.

It is within the confines of the present invention that the formulations of the IP3R agonist or the IP3R antagonist may be further associated with a pharmaceutically-acceptable carrier, thereby comprising a pharmaceutical composition. The pharmaceutically-acceptable carrier must be “acceptable” in the sense of being compatible with the other ingredients of the composition and not deleterious to the recipient thereof. Examples of acceptable pharmaceutical carriers include, but are not limited to, carboxymethyl cellulose, crystalline cellulose, glycerin, gum arabic, lactose, magnesium stearate, methyl cellulose, powders, saline, sodium alginate, sucrose, starch, talc and water, among others. Formulations of the pharmaceutical composition may conveniently be presented in unit dosage.

The formulations of the present invention may be prepared by methods well-known in the pharmaceutical art. For example, the active compound may be brought into association with a carrier or diluent, as a suspension or solution. Optionally, one or more accessory ingredients (e.g., buffers, flavoring agents, surface active agents and the like) also may be added. The choice of carrier will depend upon the route of administration. The pharmaceutical composition would be useful for administering the therapeutic agents of the present invention (i.e., the IP3R agonist and the IP3R and their analogues and derivatives, either in separate, individual formulations or in a single, combined formulation) to a subject to treat heart failure. The therapeutic agents are provided in amounts that are effective to treat or prevent heart failure in the subject. These amounts may be readily determined by the skilled artisan.

The effective therapeutic amounts of the IP3R agonist and the IP3R antagonist will vary depending on the particular factors of each case, including the particular disease being treated or prevented and the method of administration. The appropriate effective therapeutic amounts of the IP3R agonist and the IP3R antagonist within the listed ranges can be readily determined by the skilled artisan.

The present invention provides a method for preventing cell death in a subject comprising administering to a subject in need thereof a therapeutically effective amount of an IP3R antagonist, wherein the antagonist prevents or inhibits phosphorylation of IP3R1. In one embodiment, the invention provides a method for treating or preventing a disease involving apoptosis or cellular death in a subject comprising administering to a subject a therapeutically effective amount of an IPR1 antagonist. In another embodiment, the disease treated or prevented in a neurodegenerative disease such as Alzheimer's disease. The neurodegenerative diseases treated or prevented by the present invention include, but are not limited to, Alzheimer's disease, Parkinson's disease, Huntington's disease, amytrophic lateral sclerosis, Pick's disease, progressive supraneuclear palsy and coricobasal degeneration.

The invention also provides a method for treating or preventing HIV or AIDS by administering to a subject in need thereof a therapeutically effective amount of a composition comprising an IP3R antagonist.

In an additional embodiment, the present invention provides a method for inducing or increasing cellular death or apoptosis comprising administering to the cell an effective amount of a composition comprising an IP3R agonist such that the IP3R agonist improves or enhances phosphorylation of IP3R1. In a particular embodiment, the cell is a tumor cell.

The present invention further provides a method for treating or preventing a proliferative disease comprising administering to a subject in need thereof a therapeutically effective amount of a composition comprising an IP3R1 agonist such that the agonist enhances phosphorylation of IP3R1. In one embodiment, the proliferative disease is cancer.

The present invention also provides kits for use in regulating intracellular calcium and treating and preventing various disorders, diseases and disease processes. In one embodiment, the invention provides a kit for use in treating or preventing a neurodegenerative disease which includes a composition comprising an effective amount of an IP3R1 antagonist. In another embodiment, the invention provides kits for use in treating and preventing HIV infection comprising an effective amount of an IP3R1 antagonist. In yet another embodiment, the present invention provides a kit for use in treating and preventing cancer which includes a composition comprising an effective amount of an IP3R1 agonist.

Treating an individual disease process, as used herein, refers to treating any one or more of the conditions underlying the particular disease or disease process. As used herein, preventing an individual disease process includes preventing the initiation of the disease or disease process, delaying the initiation of the disease or disease process, preventing the progression or advancement of the disease or disease process, slowing the progression or advancement of the disease or disease process, delaying the progression or advancement of the disease or disease process and reversing the progression of the disease or disease process from an advanced to a less advanced stage.

In one embodiment of the invention, Alzheimer's disease is treated or prevented in a subject in need of treatment by administering to the subject a therapeutically effective amount of an IP3R antagonist effective to treat the Alzheimer's disease. The subject is preferably a mammal (e.g., humans, domestic animals, and commercial animals, including cows, dogs, monkeys, mice, pigs and rats) and is most preferably a human. The terms “therapeutically effective amount” or “effective amount” as used herein mean the quantity of the composition according to the invention which is necessary to prevent, cure, ameliorate or at least minimize the clinical impairment, symptoms or complications associated with the disease in either a single or multiple dose. The amounts of IP3R agonist and IP3R antagonist effective to treat the particular disease or disease process will vary depending on the particular factors of each case, including the stage or severity of the disease or disorder, the subject's weight, the subject's condition and the method of administration. The skilled artisan can readily determine these amounts.

### EXAMPLES

The following examples illustrate the present invention and are set forth to aid in the understanding of the invention and should not be construed to limit in any way the scope of the invention as defined in the claims which follow thereafter.

### Example 1

### Methods

Cell Culture and Reagents

The human leukemic T cell line, Jurkat cells (Clone E6.1 from the American Type Culture Collection), was cultured in RPMI medium containing 10% FBS and 100 units/ml penicillin and streptomycin. The cells were split every 2 days to maintain log phase cultures. Antiserum to IP3R1 was kindly provided by Dr. Greg Mignery (Loyola University, Chicago, Ill.). Human recombinant cdc2/CyB and nocodazole were obtained from CalBiochem (La Jolla, Calif.).

Generation of Phosphospecific Antibodies to IP3R1

Polyclonal antibodies were raised against two phosphopeptides (MLKIGTS*PVKEDKEA and DPQEQVT*PVKYARL) that encode the putative phosphorylation residues at Ser⁴²¹ and Thr⁷⁹⁹, respectively. The polyclonal antibodies were affinity-purified with two cycles of purification by initially passing through non-phosphorylated peptides and finally through the respective phosphorylated peptides. Titration and specificity of phosphospecific antibodies were determined by ELISA and immunoblotting.

Western Blotting, Immunoprecipitation, and In Vitro Kinase Reactions

Cells were equalized for number and lysed in ice-cold lysis buffer containing 0.5% Nonidet P-40, 25 mM HEPES, pH 7.4, 150 mM NaCl, 1 mM EDTA, 1 mM sodium orthovanadate and protease inhibitors. Cell lysates were centrifuged at 13,000× g in a microcentrifuge, and the supernatants were subjected to immunoblotting and immunoprecipitation (Frangioni and Neal 1993; Matter, et al., 1993). The membranes were blocked in TBST (20 mM Tris/HCl, pH 7.4, 0.9% NaCl, and 0.05% Tween 20) containing 5% nonfat dried milk for 1 hr followed by incubation with primary antibodies. After extensive washings, the membranes were incubated with secondary antibody conjugated to horseradish peroxidase (goat anti-rabbit IgG; Pharmingen, Calif.) in TBST containing 5% nonfat dried milk. The immunoblots were analyzed using an ECL detection system (Amersham, Piscataway, N.J.). For detecting in vivo phosphorylated IP3RI, DT40 cells were cultured in 10% FBS-RPMI medium with and without 1 μM nocodazole. Immunoprecipitations were performed with αIP3R1 antibody as described (Frangioni and Neal 1993) and the immune complexes were washed three times with ice-cold buffer containing 25 mM HEPES, pH 7.4, 150 mM NaCl, 1 μM sodium orthovanadate, 0.5% Nonidet P-40 and a cocktail of protease inhibitors containing 4-(2 aminoethyl) benzenesulfonyl fluoride, pepstatin A, E64, bestatin, leupeptin, and aprotinin (Sigma Biochemicals, St. Louis, Mo.). The kinase assays were performed at 30° C. for 10 min in a 25-μl volume containing 50 mM Tris, pH 7.4, 10 mM MgCl₂, 1 mM DTT, and 10 μCi of [γ-³²P] ATP with and without exogenous cdc2/CyB. Phosphoproteins were separated by SDS-PAGE and detected by autoradiography as described (Frangioni and Neal 1993; Means 1994).

Generation of Wild-Type and cdc2 Phosphorylation-Deficient Mutant GST Proteins

The generation of pGEX vectors that encode glutathione S-transferase (GST) fusion proteins and the purification of the expressed proteins have been described previously (Frangioni 1993). The regions corresponding to residues 375-473, 753-886, and 1-900 of mouse IP3R1 were amplified by PCR and cloned into pGEX2T into BamHl and EcoRl sites. S421A and T799A mutations were introduced using the Quik Change mutagenesis kit (Strategene, Calif.) and mutations were confirmed by sequencing. GST fusion constructs containing residues 375-473 (IP3R1/375-473) and 753-886 (IP3R1/775-886) as well as the mutant constructs (S421A and T799A) were expressed in JM101. Fifteen ml of cell culture (A₆₀₀=0.5) was induced with 0.1 mM isopropyl-β-D-thiogalactopyronoside (IPTG) at 37° C. for IP3R1/375-473 and at 12° C. for IP3R1/775-886 fusion proteins. Fusion proteins were purified on glutathione-agarose beads as per manufacturer's instructions (Amersham Pharmacia Biotech) and washed three times in PBS-1% Triton X-100 to remove nonspecific bound proteins.

[³IP3]IP3-Binding Assay

IP3-binding assay was performed using the IP3R1(1-900) fragment essentially as described (Zhu, et al., 1996). Soluble protein (30 μg) was incubated with 9.6 nM [³IP3] in 100 μl of binding buffer for 10 min at 4° C. The mixture was then added to 4 l of γ-globulin (50 mg/ml) and 100 μl of solution containing 30% (w/v) polyethylene glycol 6000, 50 mM Tris-HCl (pH 8.0 at 4° C.), 1 mM 2-mercaptoethanol, and 1 mM EDTA. After incubation at 4° C. for 5 min, the protein-polyethylene glycol complex was collected by centrifugation at 10,000×g for 5 min at 2° C. The pellets were dissolved in 180 μl of Solvable (DuPont NEN). After neutralization with 18 μl of acetic acid, radioactivity was measured in 5 ml of Atomlight (Dupont NEN) in a liquid scintillation counter. The specific binding was determined by subtracting the nonspecific binding in the presence of 2 μM IP3 from the total binding.

### Results

IP3R1 is a Target for cdc2/CyB In Vitro

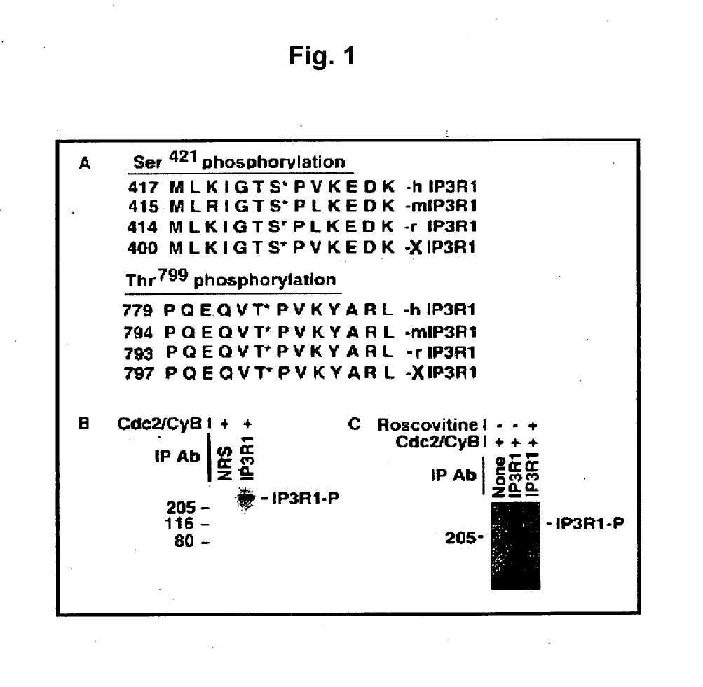

To understand the decreased proliferation of IP3R-deficient cells at a molecular level, the IP3R1 primary sequence for motifs that could influence cellular proliferation were analyzed. The inventor specifically looked for a consensus cdk-phosphorylation motif, (S/T)PX(K/R). IP3R1 is a 308-kD polypeptide that contains two putative cdk-phosphorylation sites, at residues 421 (Ser⁴²¹) and 799 (Thr⁷⁹⁹). In IP3R1, Ser⁴²¹ is within the IP3-binding domain and Thr⁷⁹⁹ is proximal to the IP3-binding domain (based on primary structure). Interestingly, the phosphorylation sites in IP3R1 are conserved from Xenopus to human (FIG. 1A). To determine if IP3R1 may function as a cdc2/CyB substrate, the inventor performed in vitro kinase reactions on immunoprecipitates of IP3R1 from brain microsomes with human cdc2/CyB complex. The results show that cdc2/CyB phosphorylates IP3R1 in vitro and that this phosphorylation is completely inhibited by the inclusion of roscovitine, a potent cdc2 inhibitor (FIG. 1C). No false-positive phosphorylation signals were detected in the kinase assays, either from beads or from immunoprecipitates made with normal rabbit serum (NRS) (FIGS. 1B and C).

Identification of cdc2 Phosphorylation Sites

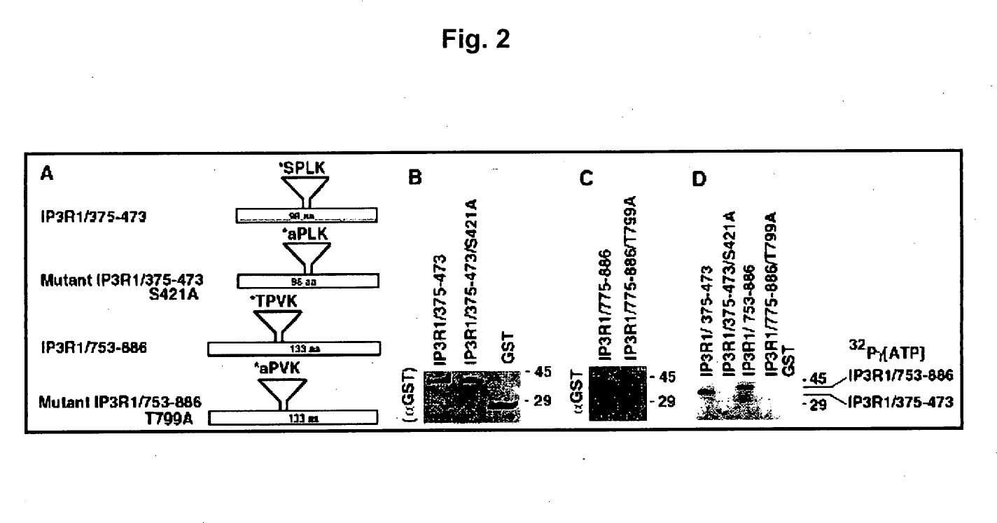

To determine the site(s) of IP3R1 phosphorylation by cdc2/CyB and their specificity, GST fusion proteins containing the putative cdk phosphorylation sites [site 1 (IP3R1/375-473) and 2 (IP3R1/753-886), and their mutants (421^{S->A} and 799^{T->A})] were generated (FIG. 2A). Both wild-type IP3R1/375-473 (FIG. 2B) and IP3R1/753-886 (FIG. 2C) and their mutants expressed equally well. Purified GST was included as a negative control. In vitro kinase reactions were performed with 200 ng of GST fusion proteins, gamma ATP and 2 units of cdc2/CyB. The phosphorylated proteins were size-fractionated on 12% SDS-PAGE gels and detected by autoradiography. Both GST fragments containing Ser⁴²¹ and Thr⁷⁹⁹ were phosphorylated by cdc2/CyB (FIG. 2D). These results suggest that cdc2/CyB phosphorylates both residues (S⁴²¹ and T⁷⁹⁹) in vitro and that the mutation of these residues to alanine abrogates phosphorylation (FIG. 2D, lanes 2 and 4).

Generation of Phosphospecific IP3R1 Antibodies

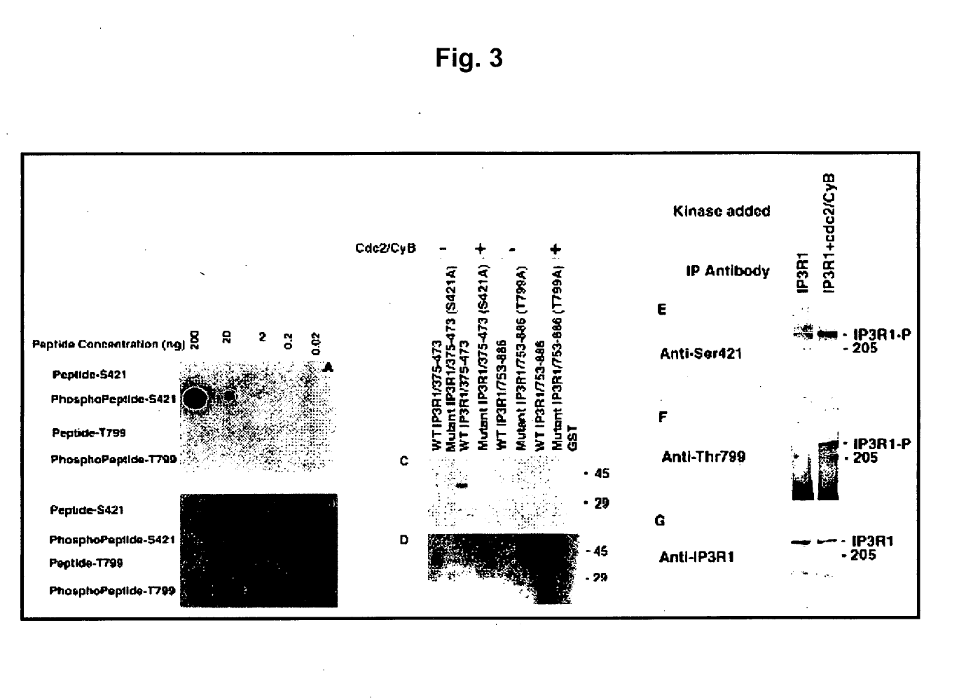

To study cdk-mediated phosphorylation of IP3Rs, two phosphospecific polyclonal antibodies that recognize the phosphorylated forms of Ser⁴²¹ and Thr⁷⁹⁹ in IP3R1 were generated. The affinity-purified antibodies were tested by dot-blot analysis using nonphosphorylated and phosphorylated peptides. The results show that both antibodies react only with their respective antigenic phosphopeptides (FIGS. 3A and B). There was no reactivity with non-phosphorylated peptides. To further characterize these antibodies, the inventor generated GST fusion proteins that contained one of the Ser⁴²¹ and Thr⁷⁹⁹ phosphorylation sites for cdks and mutant GST fusion proteins in which the Ser⁴²¹ and Thr⁷⁹⁹ were replaced with alanine. These fusion proteins were immunoblotted with αSer⁴²¹ (FIG. 3C) and αThr⁷⁹⁹ (FIG. 3D) antibodies. Again, αSer⁴²¹ strongly reacted only with the wild-type IP3R1/375-473 protein phosphorylated by cdc2/CyB, and αThr⁷⁹⁹ recognized only the wild-type fusion protein IP3R1/753-886 phosphorylated by cdc2/CyB. S421A and T799A mutations completely abolished reactivity with the respective antibodies. Importantly, these antibodies recognized only their respective phosphorylated proteins. These results clearly indicate that the antibodies are specific and recognize the phosphorylated Ser⁴²¹ and Thr⁷⁹⁹ residues in IP3R1.

To determine whether these phosphospecific antibodies react with the native IP3Rs, the inventor immunoprecipitated IP3R1 from Jurkat cells, performed kinase reactions in the absence and presence of cdc2/CyB, and immunoblotted with αSer⁴²¹ (FIG. 3E), αThr⁷⁹⁹ (FIG. 3F) and αIP3R1 antibodies (FIG. 3G). Interestingly, both αSer⁴²¹ and αThr⁷⁹⁹ recognized phosphorylated native IP3R1. A very faint band seen with the IP3R1 immunoprecipitate suggests a basal level of endogenous phosphorylation. Exogenous addition of cdc2/CyB increased the steady-state phosphorylation of IP3R1 with increased antibody reactivity/signals (FIGS. 3E and F) without any change in IP3R1 levels (FIG. 3G).

IP3R1 Phosphorylation Occurs at Ser⁴²¹ and Thr⁷⁹⁹ In Vivo

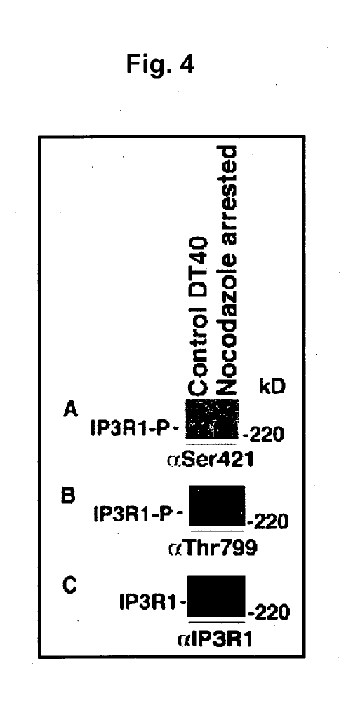

To determine whether IP3R1 phosphorylation occurs in vivo, DT40 B cells were serum starved for 24 h, then cultured them with and without 1 μM nocodazole in 10% FBS-RPMI medium for 16 h, lysed the cells, and immunoblotted cell lysates with αSer⁴²¹ (FIG. 4A), αThr⁷⁹⁹ (FIG. 4B), or αIP3R1 (FIG. 4C) antibodies. Nocodazole treatment, which arrests cells at G2/M phase with increased cdc2 activity, greatly enhanced IP3R1 phosphorylation at both Ser⁴²¹ and Thr⁷⁹⁹ residues without affecting total IP3R1 protein expression as compared to control cells (FIG. 4C). These data suggest that cdc2-mediated IP3R1 phosphorylation occurs in vivo.

Cdc2/CyB Phosphorylation Increases IP3R1's Affinity for IP3

A previous study has shown that IP3 binding to purified IP3R1 from cerebellum is stoichiometric, and that affinity residues 1-604 of mIP3R1 for IP3 were comparable to that of the native cerebellar IP3R (83 nM) (Yoshikawa, et al., 1999). Since the IP3-binding specificity of the N-terminal IP3R1 fragment expressed in E. coli was very similar to that of the native IP3R from mouse cerebellum, this approach to investigate the phosphorylation on IP3 binding was employed. A GST fragment containing the first 900 N-terminal amino acids of mIP3R1 [IP3R1(1-900)], which encode the IP3 binding pocket and the two cdc2/CyB phosphorylation sites was generated. To directly determine whether cdc2/CyB phosphorylation modulates IP3 binding, an in vitro kinase reaction with purified cdc2/CyB (FIG. 4A) followed by IP3 binding as described (Yoshikawa, et al., 1996) was performed.

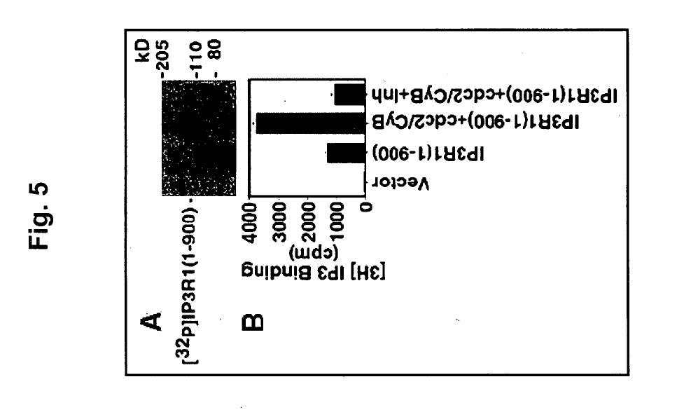

Phosphorylation of this ˜126-kD protein was observed only with the addition of cdc2/CyB and was completely inhibited by roscovitine, a cdc2 inhibitor (FIG. 5A). The phosphorylated protein fraction had approximately three-fold higher IP3 binding as compared with non-phosphorylated and cdc2-inhibitor treated fractions. The vector-alone fraction showed very little IP3 binding (FIG. 5B). These experiments indicate that cdc2 phosphorylation increases IP3 binding to IP3R1.

### Discussion

The inventor has demonstrated for the first time that IP3R1 is a target for cdc2/CyB complex in vitro and in vivo and that cdc2 phosphorylation of IP3R1 leads to increased IP3 binding in vitro.

Initial in vitro phosphorylation studies were carried out using dog cerebellum because: 1) IP3R1 is expressed at a high level (>99%) with very little IP3R2 and IP3R3; and 2) the near-exclusive expression of IP3R1 in cerebellum permits easy detection of IP3R1 phosphorylation unambiguously, as the presence of other isoforms would confound the phosphorylation analysis due to heterotetramer formation (Joseph, et al., 1995; Nucifora, et al., 1996). While these results clearly demonstrate IP3R1 phosphorylation by cdc2/CyB in vitro, the blots from DT40 cells arrested with nocodazole demonstrate that IP3R1 phosphorylation occurs in vivo (FIGS. 4A and B). Although untested, based on these results it is conceivable that cdk2 also could phosphorylate IP3R1, because both cdk1 and 2 demonstrate broad specificity over substrates containing Ser/Pro or Thr/Pro sequence motifs (Holmes 1996) and are blocked by roscovitine.

Analysis of the sequences of other IP3Rs suggests that the Thr⁷⁹⁹ phosphorylation site is conserved in IP3R3, while neither the Ser⁴²¹ or Thr⁷⁹⁹ phosphorylation site is conserved in IP3R2. In IP3R1, both cdk-phosphorylation motifs are remarkably conserved from Xenopus to human. The fact that the clustered cdk-phosphorylation sites are conserved in other species is consistent with cdk-mediated IP3R phosphorylation potentially being a conserved mechanism for modulating intracellular Ca²⁺ release during cell cycle progression.

A complete analysis of the physiological roles of cdc2-mediated IP3R1 phosphorylation has been hampered by the lack of reagents that specifically recognize the phosphorylated state of the protein. The inventor has generated phosphospecific antibodies that recognize phosphorylated Ser⁴²¹ and Thr⁷⁹⁹ in IP3R1 and demonstrated that the protein's antigenicity is abolished by mutations of Ser and Thr residues to alanine in IP3R1. The generation of these antibodies facilitated the detection of native IP3R1 protein phosphorylated by the cdc2/CyB complex in vitro (FIGS. 3E and F) and in vivo. These results suggest that the antibodies recognize site-specific phosphorylation of IP3R1 and can thus be used to further investigate the implications of this phenomenon.

IP3 mediates Ca²⁺ release from ER after binding to its receptor, IP3Rs. In IP3Rs, the IP3-binding core consists of amino acid regions 1-225 and 226-604 in the N-terminal portion of the IP3R channel (Yoshikawa, et al., 1996). Previous studies have shown that the N-terminal (amino acids 1-225) portion of the molecule acts as a suppressor of IP3 binding. The binding of at least two IP3 molecules to a single tetrameric IP3R channel is required for channel opening, and IP3 binding elicits a large conformational change in the N-terminal portion of IP3R1. These results show a three-fold increase in IP3 binding to IP3R1(1-900) upon phosphorylation by cdc2 (FIG. 4). This increase may be due to conformational changes in IP3R1 upon phosphorylation, or because phosphorylated IP3R1 may no longer be susceptible to suppression elicited by the N-terminal 225 amino acids. Additional experiments are required to test this possibility.

Independent studies have shown that: 1) cytosolic Ca²⁺ is increased during the G2/M phase or just after exit from mitosis; and 2) cdc2/CyB activity controls the generation of sperm-triggered Ca²⁺ oscillations in oocytes during the cell cycle (Deng 2000; Levasseur 2000; Tokmakov, et al., 2001). Although these studies support the fact that Ca²⁺ oscillations occur during cell cycle transitions, the importance of IP3-gated Ca²⁺ release for cellular proliferation remains controversial. For instance, while IP3R1-deficient Jurkat cells display reduced cell growth (Jayaraman and Marks 1997), T cells from an IP3R1-deficient mouse proliferate equally well as compared to T cells from the wild-type (Hirota, et al., 1998). The discrepancy between the results reported for primary T cells from IP3R1-deficient mice and for transformed Jurkat T cell lines might be due to the presence of other IP3R subtypes, which could compensate for the loss of IP3R1 function. IP3R1-deficient Jurkat lymphocytes express other IP3R subtypes at greatly reduced levels (Jayaraman and Marks 1997; Lee, et al., 2003), while IP3R1-deficient mouse T cells express other IP3R subtypes to the same levels as in the wild-type. In addition, it is conceivable that primary and transformed cells differ in their cellular effector responses upon activation. Upon activation by TCR and mitogens, primary T cells proliferate while Jurkat cells undergo apoptosis. Thus it is likely that additional Ca²⁺-dependent and Ca²⁺-independent events might be activated differently in primary and transformed cells. Alternatively, other Ca²⁺-channels and/or pumps that maintain Ca²⁺ homeostasis could compensate for the lack of IP3R1 in IP3R1-deficient T cells to sustain cellular proliferation.

Cdc2 is inactivated in normally progressing cells to allow mitosis to proceed via CyB degradation (Means 1994; Morgan 1997). However, cdc2 activation is also linked to some forms of cellular apoptosis in several pathological conditions. For instance, premature or inappropriate activation of cdc2 and increased IP3-gated intracellular Ca²⁺ release have been causally linked to pathogenesis of HIV and Alzheimer's disease (Vincent, et al., 1997; Haughey, et al., 1999; Castedo, et al., 2002). Cdc2 activity is also increased in peripheral blood mononuclear cells (PBMCs) from HIV-1 infected patients, a phenomenon that has been attributed to T cell activation, and therapeutic inhibition of HIV-1 replication decreases the expression of CyB in PBMCs (Fotedar, et al., 1995; Piedimonte, et al., 1999; Cannavo, et al., 2001). Inhibition of cdc2 activity by roscovitine or olomoucine also prevented syncytial cell death induced by HIV infection (Castedo, et al., 2002). If IP3R phosphorylation and modulation occurs in vivo in a manner similar to the in vitro results, it is possible that this regulation could play a substantial role in many pathophysiological conditions. Following this hypothesis, it is further tempting to speculate that the kinetics, magnitude and spatial and temporal localization of phosphorylated IP3Rs could lead to different cellular functions (Dolmetsch, et al., 1997). In this regard, the availability of the present phosphospecific antibodies may help in investigating the IP3R1-mediated Ca²⁺-signaling events during cell cycle progression.

### Example 2

The benefits of a therapy involving a pharmaceutical composition comprising an IP3R1 antagonist for treating neurodegenerative diseases such as Alzheimer's disease can be demonstrated in a rodent model of Alzheimer's disease.

An established rat model if Alzheimer's can be used and three groups of young adult rats can then be studied: (1) Alzheimer's+IP3R1 antagonist; (2) Alzheimer's without IP3R1 antagonist; and (3) Alzheimer's+placebo treatment. The rats' ability in performing learning and memory tasks can be tested and behavior can be directly monitored. After approximately 10 weeks of therapy, rat brains can be harvested and analyzed.

It is expected that the results of the study will demonstrate the general benefits of therapy utilizing IP3R1 antagonist for treatment of Alzheimer's disease. Rats from groups 2 and 3 should demonstrate significantly impaired ability in performing learning and memory tasks compared with group 1 rats. It is also expected that the cerebral cortex from group 2 and 3 rats should show increased evidence of neuronal death, e.g., an excessive amount of neurofibrillary tangles and neuritic or senile plaques typically associated with Alzheimer's pathogenesis. Accordingly, rats treated with IP3R1 antagonist (group 1 rats) will demonstrate the most improved behavioral parameters and least evidence of premature neuronal death as compared to Alzheimer's rats receiving placebo (group 3) or no IP3R1 antagonist treatment (group 2).

### Example 3

The benefits of a therapy involving a pharmaceutical composition comprising an IP3R1 agonist for treating tumors can also be demonstrated in a mouse tumor model.

Young adult mice can be injected with a tumor, e.g., a mouse sarcoma MCA205 or mouse melanoma B16B16, and three groups of rats can then be studied: (1) Tumor+IP3R1 agonist; (2) Tumor without IP3R1 agonist; and (3) Tumor+placebo treatment. Tumor growth can be monitored throughout the approximately 6 week period of therapy.

It is expected that the results of the study will demonstrate the general benefits of therapy utilizing IP3R1 agonist for treatment of tumor cells. Mice from group 1 should demonstrate a significant reduction in the growth rate of tumors as compared to mice from groups 2 and 3. Accordingly, rats treated with IP3R1 agonist (group 1 mice) will demonstrate the most improved general health parameters and least evidence of tumor growth as compared to mice receiving placebo (group 3) or no IP3R1 agonist treatment (group 2).

All publications referenced herein are hereby incorporated in their entirety. While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A pharmaceutical composition for use in increasing intracellular calcium comprising a therapeutically effective amount of an IP3R1 agonist.

2. A pharmaceutical composition for use in decreasing intracellular calcium comprising a therapeutically effective amount of an IP3R1 antagonist.

3. The composition of claim 1, wherein the agonist enhances phosphorylation of IP3R1 at Ser⁴²¹ and Thr⁷⁹⁹.

4. The composition of claim 2, wherein the antagonist prevents the phosphorylation of IP3R1 at Ser⁴²¹ and Thr⁷⁹⁹.

5. The composition of claim 1, wherein the agonist enhances CyB binding to IP3R1.

6. The composition of claim 2, wherein the antagonist prevents CyB binding to IP3R1.

7. The composition of claim 5, wherein the agonist enhances CyB binding to IP3R1 at Arg³⁹¹, Arg⁴⁴¹ and Arg⁸⁷¹.

8. The composition of claim 6, wherein the antagonist prevents CyB binding to IP3R1 at Arg³⁹¹, Arg⁴⁴¹ and Arg⁸⁷¹.

9. A method for preventing cell death comprising administering to cells an effective amount of an IP3R1 antagonist, wherein the antagonist prevents or inhibits phosphorylation of IP3R1.

10. A method for treating or preventing cell death in a subject comprising administering to a subject a therapeutically effective amount of an IP3R1 antagonist, wherein the antagonist prevents or inhibits phosphorylation of IP3R1.

11. A method for treating or preventing a disease involving cell death in a subject comprising administering to a subject a therapeutically effective amount of an IP3R1 antagonist, wherein the antagonist prevents or inhibits phosphorylation of IP3R1.

12. The method of claim 11, wherein the disease is a neurodegenerative disease.

13. The method of claim 12, wherein the neurodegenerative disease is a disease selected from the group consisting of: Alzheimer's disease, Parkinson's disease, Huntington's disease, amyotrophic lateral sclerosis, Pick's disease, progressive supranuclear palsy, and corticobasal degeneration.

14. The method of claim 11, wherein the disease is HIV or AIDS.

15. A method for inducing cell death comprising administering to the cell an effective amount of an IP3R1 agonist, wherein the agonist enhances phosphorylation of IP3R1.

16. The method of claim 15, wherein the cell is a tumor cell.

17. A method for treating or preventing a proliferative disease comprising administering to the subject a therapeutically effective amount of a composition comprising an IP3R1 agonist, wherein the agonist enhances phosphorylation of IP3R1.

18. The method of claim 17, wherein the proliferative disease is cancer.

19. A kit for use in treating and preventing neurodegenerative disease comprising an effective amount of an IP3R1 antagonist.

20. A kit for use in treating and preventing HIV infection comprising an effective amount of an IP3R1 antagonist.

21. A kit for use in treating and preventing cancer comprising an effective amount of an IP3R1 agonist.
